(19) United States
(12) Patent Application Publication
(10) Pub. No.: US 2009/0187126 A1
(43) Pub. Date: Jul. 23, 2009

(54) APPARATUS AND METHOD OF PROTECTING A CAST ON A HUMAN LIMB

(76) Inventor: Kari Nelson, Green Bay, WI (US)

Correspondence Address: MICHAEL BEST & FRIEDRICH LLP, 100 E WISCONSIN AVENUE, Suite 3300, MILWAUKEE, WI, 53202, US

(21) Appl. No.: 12/017,160

(22) Filed: Jan. 21, 2008

(51) Int. Cl. A61F 5/00

(52) U.S. Cl. 602/3; 602/36

(57) ABSTRACT

A protective cover for a limb cast. The protective cover includes a first layer, a second layer, a first fastener, and a second fastener. The first layer has a first thickness, a first length approximating a length of the limb cast and defines a first portion of the protective cover. The second layer encapsulates the first layer and is coupled to the first layer, the second layer having a second thickness less than the first thickness, the second layer having a second length extending beyond the first length, a difference between the first length and the second length defining a second portion of the protective cover, the second portion extending beyond the length of the limb cast, the second layer including a first end and a second end. The first cooperating fastener is secured to the second layer and is positioned within the first portion to couple the first end and the second end of the second layer together. The second cooperating fastener is secured to the second layer and is positioned within the second portion to secure the protective cover to the limb with the cast.

### BACKGROUND

Athletes of all ages, through the physical demands of their sports, often sustain injuries such as ligament sprains or fractures. When these severe injuries occur, a protective cast or splint may be applied by a healthcare provider to ensure proper healing and protection of the injury. For example, if someone were to fracture their forearm, wrist, or hand, they most likely would be put in a short arm cast.

A cast is generally a shell, frequently made from plaster or fiberglass, which encases a limb to hold a broken bone (or bones) in place until it has healed. Plaster casts include a cotton bandage that has been impregnated with plaster of paris, which hardens after water has been added. Fiberglass casts include a knitted fiberglass bandage impregnated with polyurethane, and is lighter and dries much faster than a plaster cast.

Years ago, the application of a plaster or fiberglass cast on a fractured limb would sideline an athlete from their particular sport for 4-6 weeks, which is the average time a bone fracture takes to heal. Athletes were not allowed to participate in activity, since their casted appendage could be used as a weapon and cause harm to other athletes or even themselves while participating in the sport activity.

### SUMMARY

Generally, the same rules of protection apply in today's athletics, but new provisions have been made to the rules to allow for injured athletes to participate safely. The rules for most sports typically state that an athlete wearing an arm cast can participate in competition if they have a written notice from their physician and have the cast completely covered by a minimum of ½″ (one-half inch) padding.

The cast cover of the present invention satisfies the regulations set by organizations such as the national federation of high school sports, the NCAA, and even professional sports. It is functional, easy to apply, waterproof, durable, and washable. It comes in different sizes and styles to fit the cast and also is available in a variety of colors.

In one embodiment, the invention provides a protective cover for an arm cast. The protective cover includes a first layer having a first thickness, a first length approximating a length of the limb cast and defining a first portion of the protective cover. The protective cover also includes a second layer encapsulating the first layer and coupled to the first layer, the second layer having a second thickness less than the first thickness, the second layer having a second length extending beyond the first length, a difference between the first length and the second length defining a second portion of the protective cover, the second portion extending beyond the length of the limb cast, the second layer including a first end and a second end. The protective cover further includes a first cooperating fastener secured to the second layer and positioned within the first portion to couple the first end and the second end of the second layer together, and a second cooperating fastener secured to the second layer and positioned within the second portion to secure the protective cover to the limb with the cast.

In another embodiment, the invention provides a protective cover for an arm cast. The protective cover includes a body and a plurality of fasteners. The body has a length approximating a length of the arm cast and a width sufficient to wrap around the arm cast. The body includes a first layer having a length and a width substantially similar to the length and width of the body, the first layer having a first thickness and a first surface and a second surface, and a second layer covering the first surface and the second surface of the first layer, the second layer having a second thickness less than the first thickness, the second layer having a first end and a second end. The plurality of fasteners are positioned along the length of the body, a first portion of the fastener is secured to one of the first end and the second end of the second layer and a second cooperating portion of the fastener is secured to the opposite one of the first end and the second end of the second layer. The first portion of the fastener and the second cooperating portion of the fastener are adapted to be coupled together to couple the first end of the second layer to the second end of the second layer.

Other aspects of the invention will become apparent by consideration of the detailed description and accompanying drawings.

### BRIEF DESCRIPTION OF THE DRAWINGS

FIG. 1 is a perspective view of a cast applied to a human arm.

FIG. 2 is a perspective view of a cast applied to a human arm.

FIG. 3 is a perspective view of a cast cover according to one embodiment of the present invention.

FIG. 4 is a perspective view of a cast cover according to one embodiment of the present invention.

FIG. 5 is a perspective view of a cast cover according to one embodiment of the present invention.

FIG. 6 is a perspective view of a cast cover according to one embodiment of the present invention.

FIG. 7 is a perspective view of a cast cover according to one embodiment of the present invention.

FIG. 8 is a cross-sectional view of the cast cover illustrated in FIGS. 4-7.

FIG. 9 is a cross-sectional view of the cast cover illustrated in FIGS. 4-7 according to another construction of the cast cover.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

Although directional references, such as upper, lower, downward, upward, rearward, bottom, front, rear, etc., may be made herein in describing the drawings, these references are made relative to the drawings (as normally viewed) for convenience. These directions are not intended to be taken literally or limit the present invention in any form. In addition, terms such as “first”, “second”, and “third” are used herein for purposes of description and are not intended to indicate or imply relative importance or significance.

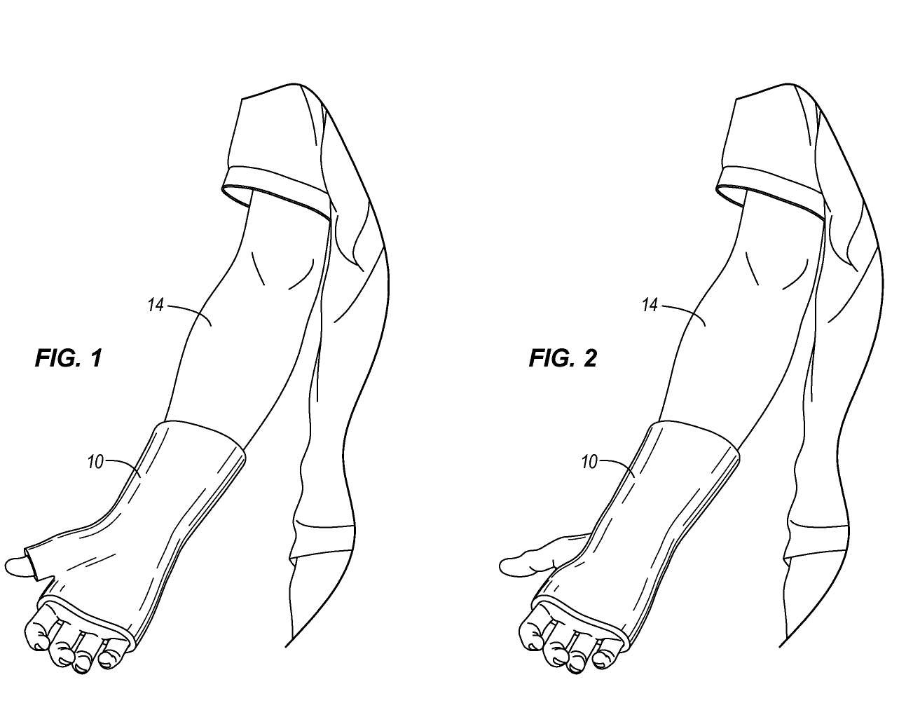

FIGS. 1 and 2 illustrate a cast 10 applied to a human arm 14. The cast 10 can include plaster or fiberglass materials applied by medical personnel to the arm 14 due to fracture of a bone(s) in the arm 14. The cast 10 is applied to set or secure the fractured bone(s) in the arm 14 for healing. FIG. 1 illustrates a cast 10 including the thumb, whereas the cast 10 illustrated in FIG. 2 does not include the thumb.

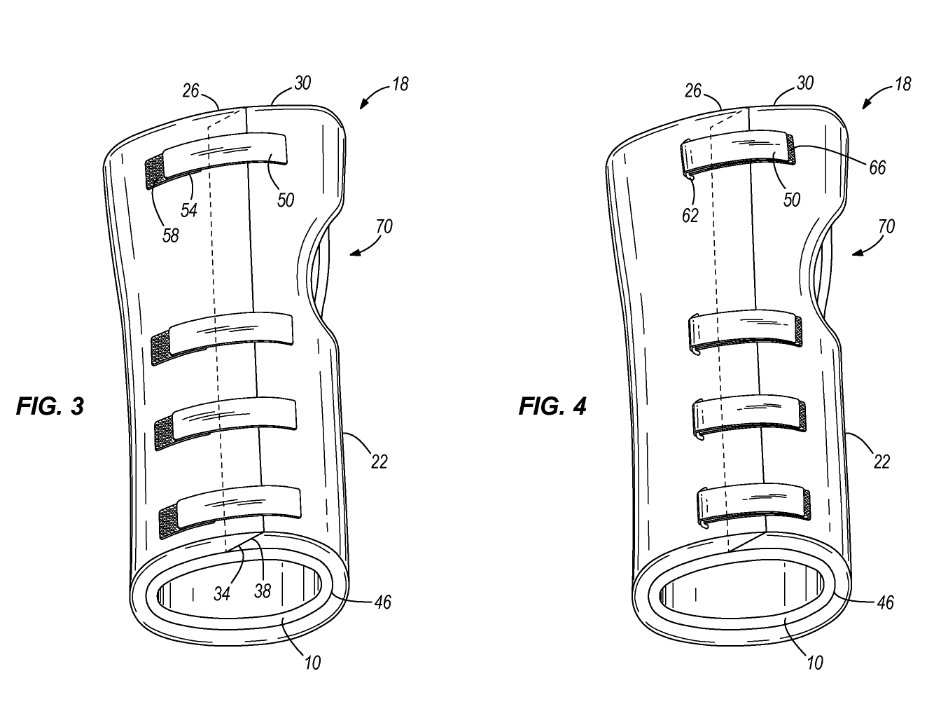

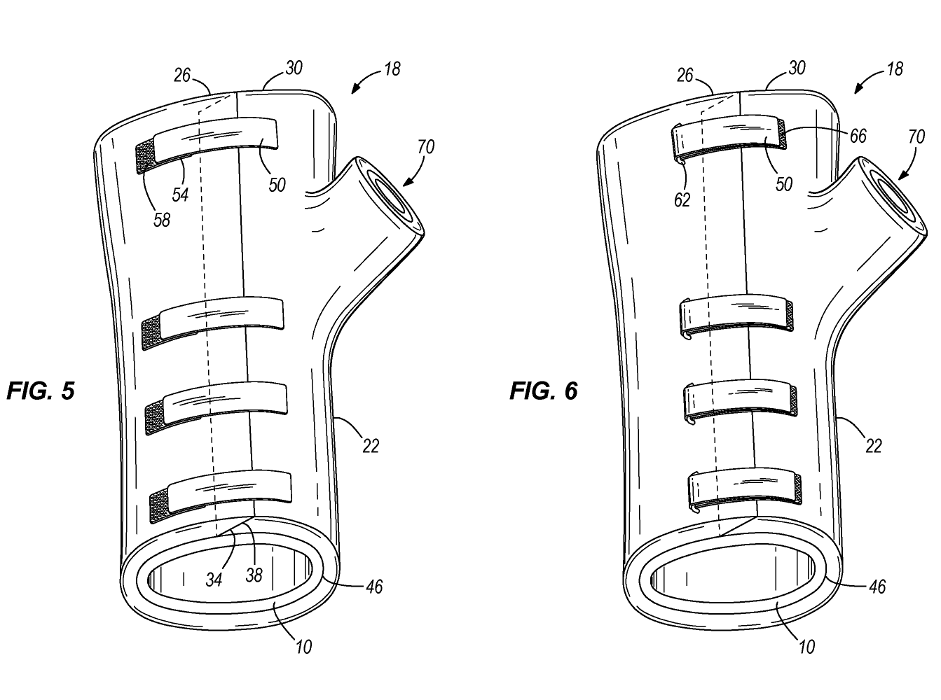

FIGS. 3-6 illustrate a protective cover 18 according to several constructions of the present invention. The protective cover 18 is applied over the cast 10, and can be applied to an arm cast. The protective cover 18 also can be modified to be applied to other casts applied to the human body.

The protective cover 18 includes a body 22 having a first end 26 and a second end 30. The first end 26 is adapted to couple to the second end 30. The first end 26 includes a sloped surface 34, and the second end 30 includes a corresponding sloped surface 38 adapted to be coupled to the sloped surface 34 of the first end 26. The sloped surface 34 and/or the sloped surface 38 can include a fastener, such as hook and loop, adhesive, snaps, or other suitable mechanism to secure the first end 26 to the second end 30. The first end 26 and the second end 30 can be comprised of the same material or different material than the body 22.

The protective cover 18 also includes a recess 46 adapted to receive the cast 10 when the protective cover 18 is wrapped around the cast 10. The first end 26 and the second end 30 are coupled together at their respective sloped surfaces 34, 38 when the protective cover 18 is wrapped around the cast 10.

The protective cover 18 also includes a plurality of straps 50 secured to the body 22 adjacent to one of the first end 26 and the second end 30. The straps 50 can be flexible and can include a fastener 54, such as the respective portion of a hook and loop fastener, adhesive, snaps, or other suitable mechanism. The protective cover 18 also includes a plurality of targets 58 positioned adjacent to one of the first end 26 and the second end 30 opposite the plurality of straps 50. The plurality of targets 58 are adapted to receive and secure the straps 50 and to retain the first end 26 adjacent to the second end 30. The targets 58 can include the respective portion of a hook and loop fastener, adhesive, snaps, or other suitable mechanism to receive and secure the straps 50.

One construction of the protective cover 18 can include a plurality of loops or buckles 62 in lieu of or in addition to the plurality of targets 58 as illustrated in FIG. 4. The loops 62 are secured to the body 22 adjacent to one of the first end 26 and the second end 30 and opposite the end 26, 30 in which the plurality of straps 50 are located. Each loop 62 is generally aligned with a corresponding strap 50. In this construction, each strap 50 includes a fastener 66, such as hook and loop, adhesive, snaps, or other suitable mechanism. For example, one end of the strap 50 includes a first portion of the fastener 66 while a second end of the strap 50 includes a second portion of the fastener, such that the first portion and the second portion are adapted to couple together. Each of the straps 50 is looped through the respective loop 62 and is folded back onto itself to couple together the first end 26 and the second end 30 of the protective cover 18.

The protective cover 18 also includes an opening 70 through the body 22 adapted to receive a thumb of the casted limb. The opening 70 can include a stiched border or edging to provide a smooth surface for the user.

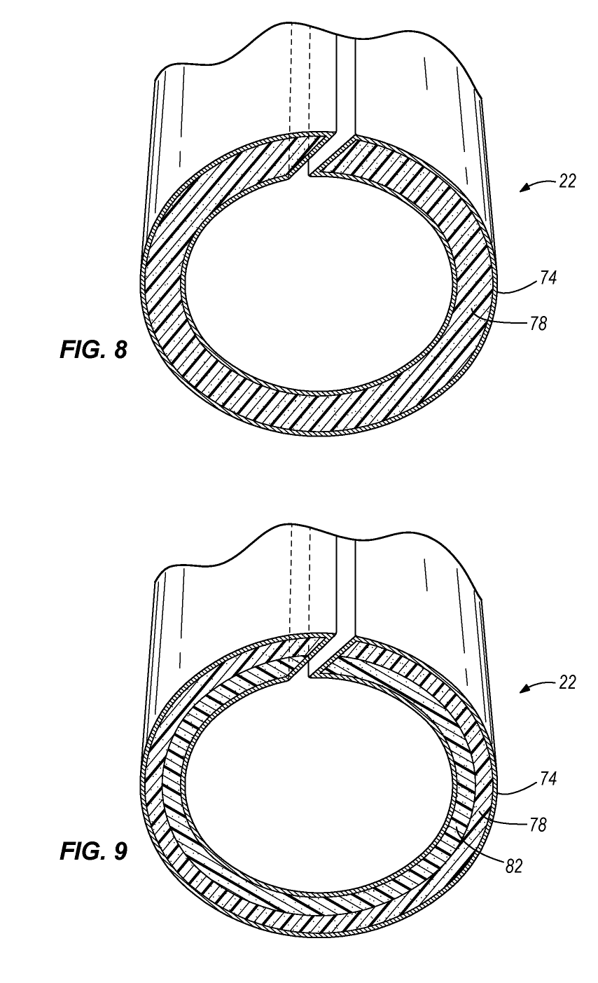

As illustrated in FIG. 8, the body 22 includes a first layer 78 positioned adjacent to a second layer 74. The first layer 78 may be secured to the second layer 74 by stitching, glue or other suitable means. The first layer 78 can extend along a portion of or a substantial length of the protective cover 18. The first layer 78 comprises a material of sufficient thickness and flexibility to pad the cast 10 and comply with the rules for participation in high school and collegiate sports, such as football. The material for the first layer 78 can comprise a foam-like material, such as a high density foam. More specifically, the high density foam can be further characterized as a closed cell polyurethane foam or slow-recovery foam. The first layer 78 can have a thickness in the range of about ⅛″ to about five inches.

The body 22 of the protective cover 18 also includes a second layer 74 comprising a material that is waterproof, easy to clean, and durable. The second layer 74 generally comprises the outer layer of the protective cover 18 and can protect the first layer 78 from damage during use and reuse. For example, the material of the second layer 74 can comprise one of or a combination of the following types and/or brands of materials: closely woven cotton, wax impregnated cotton, Polymer-Encapsulated cotton EPIC, Gore-Tex® functional textiles, Nikwax® Directional textiles, eVent waterproof fabrics, Sympatex® Technology membranes, Epoxy, 3-ply Entrant-EV membrane, Hyper-Dri, Drilite Extreme, Ventile, HyVent, Lycra Nylon, H₂NO, vinyl, Omni-Tech, neoprene, Conduit, PVC coated nylon, Triple Point Ceramic, Polyurethane coated nylon, Precip, Hydrodry P3, Watergate, Triple Point Ceramic, and Aquadry fabrics. The preferred material for the second layer 74 is vinyl because of its durability and ease of cleaning. Vinyl can withstand the applied stresses during sports activities. The second layer 74 can have a thickness in the range of about 1/32″ to about ⅛″ and includes a length and width sufficient to cover the cast 10. The second layer 74 forms a pocket and substantially encloses the first layer 78. The second layer 74 can include stitching around the second layer and/or on the first layer 78 to hold the first layer in position.

In this construction, the first layer 78 is supported within an enclosure or pocket formed by the second layer 74 such that the first layer 78 can be removed and replaced as needed. The second layer 74 includes an opening for access to the interior of the enclosure. Also in this construction, the first layer 78 can comprise a single sheet of material and/or a collection of smaller pieces of the material. For example, the first layer 78 can comprise a plurality of smaller pieces of the material (or combinations of the materials) connected together by fastener means, such as glue, heat, and/or tape. Alternatively, the first layer 78 can comprise a plurality of smaller pieces of the material (or combinations of materials) held within the enclosure or pocket to form a dense sheet-like material.

As illustrated in FIG. 9, the body 22 of the protective cover 18 also can include a third layer 82 positioned adjacent to the first layer 78. The third layer 82 can extend along a portion of or a substantial length of the first layer 78 and/or the second layer 74. The third layer 82 can comprise a material such as a layer of cotton or other suitable padding material. The third layer 82 can be secured to the first layer 78 and/or the second layer 74 by stitching, glue, hook and loop fastener, or other suitable temporary or permanent means. Alternatively, the perimeter of the third layer 82 can be secured to the perimeter of the second layer 74. In one construction the third layer 82 can have a thickness in the range of about ⅛″ to about five inches. The third layer 82 can have a thickness substantially the same as or different than the thickness of the first layer 78.

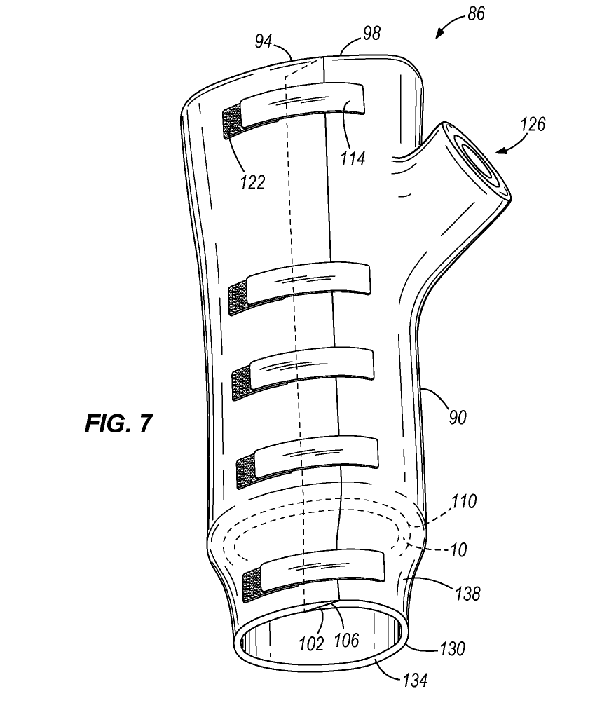

FIG. 7 illustrates a protective cover 86 according to another construction of the present invention. The protective cover 86 is applied over the cast 10, and can be applied to an arm cast. The protective cover 86 also can be modified to be applied to other casts applied to the human body.

The protective cover 86 includes a body 90 having a first end 94 and a second end 98. The first end 94 is adapted to couple to the second end 98. The first end 94 includes a sloped surface 102, and the second end 98 includes a corresponding sloped surface 106 adapted to be coupled to the sloped surface 102 of the first end 94. The sloped surface 102 and/or the sloped surface 106 can include a fastener, such as hook and loop, adhesive, snaps, or other suitable mechanism to secure the first end 94 to the second end 98. The first end 94 and the second end 98 can be comprised of the same material or different material than the body 90.

The protective cover 86 also includes a recess 110 adapted to receive the cast 10. The protective cover 86 is wrapped around the cast 10 and the first end 94 and the second end 98 are coupled together at their respective sloped surfaces 102, 106.

The protective cover 86 also includes a plurality of straps 114 secured to the body 90 adjacent to one of the first end 94 and the second end 98. The straps 114 can be flexible and can include a fastener 118, such as the respective portion of a hook and loop fastener, adhesive, snaps, or other suitable mechanism. The protective cover 86 also includes a plurality of targets 122 positioned adjacent to one of the first end 94 and the second end 98 opposite the plurality of straps 114. The plurality of targets 122 are adapted to receive and secure the straps 114 and to retain the first end 94 adjacent to the second end 98. The targets 122 can include the respective portion of a hook and loop fastener, adhesive, snaps, or other suitable mechanism to receive and secure the straps 114.

One construction of the protective cover 86 can include a plurality of loops or buckles in lieu of or in addition to the plurality of targets 122. The loops are secured to the body 90 adjacent to one of the first end 94 and the second end 98 and opposite the end 94, 98 in which the plurality of straps 114 are located. Each loop is generally aligned with a corresponding strap 114. In this construction, each strap 114 includes a fastener, such as hook and loop, adhesive, snaps, or other suitable mechanism. For example, one end of the strap 114 includes a first portion of the fastener while a second end of the strap 114 includes a second portion of the fastener, such that the first portion and the second portion are adapted to couple together. Each of the straps 114 is looped through the respective loop and is folded back onto itself to couple together the first end 94 and the second end 98 of the protective cover 86.

The protective cover 86 also includes an opening 126 through the body 90 adapted to receive a thumb of the casted limb. The opening 126 can include a stiched border or edging to provide a smooth surface for the user.

As discussed above with respect to FIGS. 8 and 9, the protective cover 86 also includes a first layer 134 (e.g., inner layer) and a second layer 130 (e.g., outer layer). The first layer 134 and the second layer 130 are substantially similar to the first layer 78 and the second layer 74, respectively as described above. The protective cover 86 includes an extended portion 138 that extends from the body 90 and is distal from the location of the hand. The extended portion 138 includes the first layer 134 and has a reduced diameter than a diameter of the first layer 78 of the body 90. As illustrated in FIG. 7, only the first layer 134 extends beyond the body 90 and forms the extended portion 138. In other constructions, the extended portion 138 may include the second layer 130. The extended portion 138 also includes a cooperating fastener such as the fasteners described above. The extended portion 138 with cooperating fastener can be secured to the arm above the cast 10 to prevent the protective cover 86 from slipping off of the cast 10.

The embodiments described above and illustrated in the figures are presented by way of example only and are not intended as a limitation upon the concepts and principles of the present invention. As such, it will be appreciated by one having ordinary skill in the art that various changes in the elements and their configuration and arrangement are possible without departing from the spirit and scope of the present invention.

What is claimed is:

1. A protective cover for a limb cast, the protective cover comprising:
a first layer having a first thickness, a first length approximating a length of the limb cast and defining a first portion of the protective cover;
a second layer encapsulating the first layer and coupled to the first layer, the second layer having a second thickness less than the first thickness, the second layer having a second length extending beyond the first length, a difference between the first length and the second length defining a second portion of the protective cover, the second portion extending beyond the length of the limb cast, the second layer including a first end and a second end;
a first cooperating fastener secured to the second layer and positioned within the first portion to couple the first end and the second end of the second layer together; and
a second cooperating fastener secured to the second layer and positioned within the second portion to secure the protective cover to the limb with the cast.

2. The protective cover of claim 1, further comprising an opening through the first layer and the second layer adapted to receive a thumb.

3. The protective cover of claim 1 wherein the second layer comprises a waterproof material.

4. The protective cover of claim 1 wherein the first layer comprises a foam material.

5. The protective cover of claim 4, wherein the first thickness is in the range of about one-eighth inch to about five inches.

6. The protective cover of claim 1, wherein the first end of the first layer has a sloped surface and the second end of the first layer has a corresponding sloped surface, and wherein the sloped surfaces of the first layer are adapted to be coupled together.

7. The protective cover of claim 6, wherein the sloped surfaces of the first layer include a cooperating fastener.

8. The protective cover of claim 1, further comprising a third layer coupled to the first layer.

9. The protective cover of claim 1, wherein the second layer forms a pocket defining an interior volume, the second layer including an opening for access to the interior volume.

10. The protective cover of claim 9, wherein the first layer is removable from the pocket.

11. The protective cover of claim 1, wherein the first cooperating fastener includes a first portion secured to the second layer and positioned adjacent the first end of the second layer, and a second corresponding portion secured to the second layer and positioned adjacent the second end of the second layer.

12. The protective cover of claim 1, wherein the second cooperating fastener includes a first portion secured to the second layer and positioned adjacent the first end of the second layer, and a second corresponding portion secured to the second layer and positioned adjacent the second end of the second layer.

13. The protective cover of claim 1, wherein the first cooperating fastener includes a loop secured to the second layer and positioned adjacent the first end of the second layer, and a strap having a first end and a second end, the first end of the strap secured to the second layer and positioned adjacent the second end of the second layer, and wherein the second end of the strap is adapted to be received through the loop and secured to the first end of the strap.

14. The protective cover of claim 1, wherein the second cooperating fastener includes a loop secured to the second layer and positioned adjacent the first end of the second layer, and a strap having a first end and a second end, the first end of the strap secured to the second layer and positioned adjacent the second end of the second layer, and wherein the second end of the strap is adapted to be received through the loop and secured to the first end of the strap.

15. A protective cover for a limb cast, the protective cover comprising:
a body having a length approximating a length of the arm cast and a width sufficient to wrap around the arm cast, the body including
a first layer having a length and a width substantially similar to the length and width of the body, the first layer having a first thickness and a first surface and a second surface, and
a second layer covering the first surface and the second surface of the first layer, the second layer having a second thickness less than the first thickness, the second layer having a first end and a second end; and
a plurality of fasteners positioned along the length of the body, a first portion of the fastener secured to one of the first end and the second end of the second layer and a second cooperating portion of the fastener secured to the opposite one of the first end and the second end of the second layer, wherein the first portion of the fastener and the second cooperating portion of the fastener are adapted to be coupled together to couple the first end of the second layer to the second end of the second layer.

16. The protective cover of claim 15 wherein the second layer comprises a waterproof material.

17. The protective cover of claim 15 wherein the first layer comprises a foam material.

18. The protective cover of claim 17, wherein the first thickness is in the range of about one-half inch to about five inches.

19. The protective cover of claim 15, wherein the first end of the second layer has a sloped surface and the second end of the second layer has a corresponding sloped surface, and wherein the sloped surfaces of the second layer are adapted to be coupled together.
